(19) United States
(12) Patent Application Publication
(10) Pub. No.: US 2023/0062537 A1
(43) Pub. Date: Mar. 2, 2023

(54) Therapy of oxygen pulses for treating neurodegenerative disorders such as Parkinson Disease (PD), Alzheimer’s Disease (AD), Amiotrophic Lateral Sclerosis (ALS) or Motor Neuron Disease (MND) and other dementias, and Lymphedema, Arthritis and Depression

(71) Applicant: jose hernandez, Santiago (CL)

(72) Inventor: jose hernandez, Santiago (CL)

(21) Appl. No.: 17/462,153

(22) Filed: Aug. 31, 2021

(51) Int. Cl. A61M 16/10; A61M 16/00; A61M 16/06

(52) U.S. Cl. CPC A61M 16/1005; A61M 16/0003; A61M 16/06; A61M 2016/003; A61M 2016/1025; A61M 2202/0208; A61M 2205/05

(57) ABSTRACT

A therapy of oxygen pulses delivered through the pulmonary system of human patient for treating neurodegenerative disorders such as Parkinson Disease (PD), Alzheimer's Disease (AD), Amiotrophic Lateral Sclerosis (ALS) or Motor Neuron Disease (MND) and other dementias, and Lymphedema, Arthritis and Depression is provided. Aspects of the methods including administering to the subjects an effective amount of oxygen as pulses through the respiratory tract are included. Also provided are methods of assessing severity of the disease, mild, moderate, severe, or critical, and oxygen doses and frequencies. The method can be applied to human patient for treating neurodegenerative disorders such as Parkinson Disease (PD), Alzheimer's Disease (AD) Amiotrophic Lateral Sclerosis (ALS) or Motor Neuron Disease (MND) and other dementias, and Lymphedema, arthritis and depression.

### FIELD OF THE INVENTION

This invention relates to medical treatments and composition and procedures useful therein. More specifically, it relates to a therapy of oxygen pulses delivered through the pulmonary system of human patient for treating neurodegenerative disorders such as Parkinson Disease (PD), Alzheimer's Disease (AD), amyotrophic lateral sclerosis (ALS) or Motor Neuron Disease (MND) and other dementias, and Lymphedema, arthritis and depression among others

### BACKGROUND OF THE INVENTION

Parkinson's disease (PD) is a neurodegenerative disorder that currently affects nearly 1 million people in the United States, although some estimates are much higher. The average age of onset is about 60 years, and as a greater proportion of the U.S. lives longer, disease prevalence is expected to increase.

Parkinson's disease progresses slowly as small clusters of neurons in the midbrain die. The gradual loss of these neurons reduces levels of a chemical, called dopamine, which is responsible for transmitting messages to the parts of the brain that coordinate muscle movement. Common symptoms include tremors or shaking in hands, arms, legs, jaw and face; rigidity or stiffness of the limbs and trunk; bradykinesia, or slowness of movement; and difficulties with balance, speech and coordination. Symptoms of Parkinson's disease begin gradually and typically worsen over time.

While scientists do not know the exact cause of Parkinson's disease, most agree that the combination of a person's genes, environment, and the interaction between these two factors play a role in disease onset and progression. By identifying the environmental exposures associated with Parkinson's disease and understanding the biological processes that dictate how the disease develops and progresses, scientists can develop approaches to prevent, diagnose, and treat the disease.

Alzheimer's disease (AD) is a complex progressive neurodegenerative disorder and the leading cause of dementia. It is estimated that close to 50 million people worldwide are affected by dementia, including AD, and the prevalence of the disease is projected to more than double by 2050.

Risk factors for AD include age, family history, apolipoprotein E ε4 genotype, diabetes, hypertension, obesity, hypercholesterolemia, traumatic brain injury, and low education level. Mutations in genes presenilin 1 (PSEN1), presenilin 2 (PSEN2), and amyloid precursor protein (APP) are associated with early-onset autosomal-dominant AD.

Alzheimer disease is complex neuropathologically and is characterized by extracellular amyloid plaques, intracellular neurofibrillary tangles, and nerve cell death. Amyloid plaques are composed of amyloid-β (Aβ), a cleavage product of the amyloid-β protein precursor (AβPP). AβPP is progressively cleaved by β-secretase (BACE 1) and then γ-secretase to form AP. AP monomers progressively aggregate into oligomers, fibrils, and insoluble amyloid plaques. Neurofibrillary tangles, the other hallmark protein aggregate in AD, are made up of hyperphosphorylated tau protein. Under normal conditions, tau promotes stabilization of microtubules; when hyperphosphorylated, tau accumulates into “tangles” composed of paired helical filaments. The amyloid cascade hypothesis of AD posits that accumulation of AP dysregulates synaptic and neuronal function, creating the intracellular conditions for formation of neurofibrillary tangles, leading to neuronal loss and further compromise of neurotransmitter function. Loss of cholinergic neurons in the basal forebrain (and consequent loss of signaling) is hypothesized to create a cholinergic deficit contributing to short-term memory loss in. These complex pathologies may occur sequentially but many are present simultaneously in the brain of the person with AD.

Although Alzheimer's disease (AD) is the world's leading cause of dementia and the population of patients with AD continues to grow, no new therapies have been approved in more than a decade. Many clinical trials of single-agent therapies have failed to affect disease progression or symptoms compared with placebo.

Despite the growing population of patients with AD, only five treatment options are currently approved to treat the cognitive symptoms of AD in the United States, the most recent of which (memantine) was approved more than a decade ago. Four of the five standard-of-care treatments are also licensed in the European Union; these include three cholinesterase inhibitors (donepezil, galantamine, and rivastigmine) and one N-methyl-D-aspartate receptor antagonist. In 2014, a fifth treatment option consisting of a fixed-dose combination with donepezil and memantine was approved for the treatment of patients with moderate to severe AD dementia who are on stable donepezil therapy. Most therapeutic agents under development over the past 15 years have failed; AD is among the least well-served therapeutic areas for drug treatments. Nearly all trials conducted to date have been monotherapy trials comparing an active agent with placebo with or without a background standard-of-care agent, such as cholinesterase inhibitors or memantine. (Cummings and Tong, 2019).

Jeffrey Cummings, Gary Tong and Clive Ballard, J Alzheimers Dis. 2019; 67(3): 779-794. Motor neuron diseases (MND)—A group of progressive neurological disorders that cause the death of the nerve cells (neurons) in the brain and spinal cord that control the muscles that enable us to do physical movements.

Motor neuron disease (MND) is also called amyotrophic lateral sclerosis (ALS) and Lou Gehrig's disease. It is a rapidly progressing, neurological disease.

MND often begins with weakness of the muscles in the hands, feet or voice, although it can start in different areas of the body and progress in different patterns and at different rates. People with MND become increasingly disabled. Life expectancy after diagnosis is one to five years, with 10 percent of people with MND living 10 years or more.

The needs of people with MND are complex and vary from person to person.

The physical effects of motor neuron disease can include: muscle aches, cramps, twitching, clumsiness, stumbling, weakness or changes in hands, arms, legs and voice, slurred speech, swallowing or chewing difficulty, fatigue, muscle wasting, weight loss, motional lability—for example, where a slight upset can cause an exaggerated response, such as crying or laughing, cognitive change (changes in thought processes), respiratory changes.

It was thought that MND only affected the nerve cells controlling the muscles that enable people to move, speak, breathe and swallow. However, it is now known that up to 50 percent of people with MND can experience changes in cognition, language, behaviour and personality. Most people experience relatively mild changes.

The causes of MND are unknown, but worldwide research includes studies on: exposure to viruses, exposure to certain toxins and chemicals, genetic factors, inflammation and damage to neurons caused by an immune system response, nerve growth factors, growth, repair and ageing of motor neurons.

Familial (hereditary) MND accounts for about five to 10 percent of cases. Several gene mutations have been identified since 1993, and current research aims to identify further genes linked to MND. The majority of cases, 90 to 95 percent, are sporadic.

People who have MIND may: develop generalized paralysis (paralysis of both sides of the body), lose speech and have difficulty swallowing, become breathless and experience sleep disturbance, experience mild cognitive and behavioral change, become increasingly dependent on others for all aspects of day-to-day activity.

MND is still incurable, but it is not untreatable, as many symptoms can be managed. The drug riluzole available on the Pharmaceutical Benefits Scheme—has been demonstrated in clinical trials to prolong survival by several months and may help people to remain in the milder phase of the disease for longer.

Research has shown that people live better and longer under the care of a multidisciplinary team. Interventions such as assistance with nutritional intake and breathing improve quality of life.

Costly and unproven therapies are sometimes recommended by well-meaning people. Seek professional advice before trying unproven therapies.

U.S. Pat. No. 11,027,000 relates to compositions and methods for the preparation, manufacture and therapeutic use of polynucleotides encoding AADC for the treatment of Parkinson's Disease.

U.S. Pat. No. 11,008,614 provides methods and materials related to genetic variations of neurological disorders. For example, this document provides methods for using such genetic variations to assess susceptibility of developing Parkinson's disease.

U.S. Pat. No. 11,007,232 is based in part methods for treating neurodegenerative diseases and disorders. Specifically, the present invention discloses methods for treating neurodegenerative disorders suing neural stem cells (NSCs) and/or pluripotent stem cell (PSC) derived neurons or neuron precursor cells. The present invention also discloses methods to induce endogenous dopaminergic neurons to release dopamine and increase the levels of dopamine in a subject.

U.S. Pat. No. 11,007,228 relates to a pharmaceutical composition containing stem cells in which vascular endothelial growth factor (VEGF) is overexpressed as an effective ingredient for preventing or treating neurodegenerative diseases. The stem cells in which VEGF is overexpressed according to the present invention effectively act against the neurodegenerative disease-induced VEGF reduction of neural stem cells in the SVC to inhibit the abnormal migration of neural stem cells, suppress inflammatory responses, and restrain the accumulation of cholesterol and spingolipids in the cerebral cortex, thus restoring the behavior exercise capacity of animal models, whereby the pharmaceutical composition can be used as an effective therapeutic agent for neurodegenerative diseases.

U.S. Pat. No. 11,001,891 provides methods for treating Parkinson's Disease (PD), e.g., PD associated with a genetic mutation in a glucocerebrosidase (GBA) gene or a leucine rich repeat kinase 2 (LRRK2) gene. The methods comprise administering to the subject a modulator, e.g., an inhibitor, of p53-inducible gene 3 (PIG3).

U.S. Pat. No. 11,001,591 relates to compounds which are suitable for the treatment of cancer, an immune disease, Parkinson's disease, Cardiac Hypertrophy or Type-2 diabetes and to pharmaceutical compositions containing such compounds. The invention further relates to a kit of parts comprising such compounds.

U.S. Pat. No. 10,973,815 provides for compositions and methods for the treatment of Parkinson's disease comprising a compound of formula (i): ##STR00001## or a pharmaceutically effective salt or ester thereof alone or in combination with L-DOPA to provide a synergistic effect, thereby providing methods of (1) treating patients with Parkinson's Disease for whom L-DOPA is no longer effective, (2) treating patients with Parkinson's Disease who developed dyskinesia due to L-DOPA, (3) treating patients with Parkinson's Disease who have are receiving deep brain stimulation and (4) treating patients with Parkinson's Disease whose symptoms interfere with activities of daily living.

U.S. Pat. No. 10,973,784 is directed to tablets for oral administration to a subject, comprising a therapeutically effective amount of SYN120 or a pharmaceutically acceptable salt thereof, wherein the tablet is substantially free of lactose. The present invention is also directed to methods for treating diseases or conditions including Alzheimer's disease and/or Parkinson's disease, comprising administering to a patient in need thereof tablets regarding the same.

U.S. Pat. No. 10,967,073 is directed, in part, to the treatment of a subject having a neurodegenerative disorder, such as Parkinson's disease (PD), by providing glucocerebrosidase enzyme. The enzyme may be provided, e.g., through gene therapy or by administration of a glucocerebrosidase protein. Accordingly, the present invention encompasses glucocerebrosidase nucleic acids or proteins for use in the treatment of PD or other neurodegenerative disorders.

U.S. Pat. No. 10,966,962 is directed to a method for treating a neurodegenerative disease such as amyotrophic lateral sclerosis (ALS), Alzheimer disease, Parkinson's disease, Huntington's disease, frontotemporal degeneration, dementia with Lewy bodies, a motor neuron disease, or a demyelinating disease. The method comprises administering to a subject in need thereof a Ppargc1a activator 2-(4-tert-butylphenyl)-1H-benzimidazole, 2-[4-(1,1-dimethylethyl)phenyl]-1H-benzimidazole, in an effective amount. A preferred route of administration is oral administration.

U.S. Pat. No. 10,946,019 provides a series of substituted aryl pyrimidine compounds and the use of these compounds as therapeutics to treat or prevent neurodegenerative disorders, including Parkinson's disease. Compounds of the invention are also able to treat the symptoms of such diseases and therefore represent a new treatment modality for ameliorating chronic and acute conditions. The compounds of the invention are capable of selectively potentiating the activity of the Nurr1:RXR.alpha. heterodimer, and are able to treat diseases or conditions associated with aberrant Nurr1:RXR.alpha. function. The invention further provides methods for treating neurodegenerative disorders by administration of Nurr1:RXR.alpha. activating agents.

U.S. Pat. No. 10,905,879 relates to a peripheral nerve stimulator that can be used to stimulate a peripheral nerve to treat essential tremor, Parkinsonian tremor, and other forms of tremor. The stimulator can have electrodes that are placed circumferentially around the patient's wrist or arm. Specific nerves in the wrist or arm can be targeted by appropriate spacing of the electrodes. Positioning the electrodes on generally opposing sides of the target nerve can result in improved stimulation of the nerve. The stimulation pattern may alternate between the nerves. Improved stimulation algorithms can incorporate tremor feedback, external data, predictive adaptation, and long-term monitoring data.

U.S. Pat. No. 10,898,710 relates to an apparatus for simulating hand tremors caused by Essential Tremor and Parkinson's disease which may also be used as part of a therapeutic program in association with surgically implanted stem cells introduced into a patient's substantia nigra.

U.S. Pat. No. 10,864,244 provides pharmaceutical compositions comprising rationally designed peptide analogs of the p65-TAD binding region of GILZ to selectively sequester activated p65. Structural and functional analyses suggest that select GILZ analog (GA) bind p65-TAD with optimum affinity, exhibit an estimated half minimal lethal dose comparable to known peptide drugs and suppress A.beta.1-42 induced cytotoxicity. Furthermore, the present disclosure provides uses and methods of using the pharmaceutical compositions, and uses and methods of using pharmaceutical formulations comprising the pharmaceutical compositions, for the treatment of neurodegenerative diseases such as Alzheimer's Disease, Parkinson's Disease, multiple sclerosis, and amyotrophic lateral sclerosis (ALS).

U.S. Pat. No. 10,857,120 provides a method for the treatment of Parkinson's disease comprising simultaneously or sequentially administering to a patient in need of treatment of Parkinson's disease a dosage form comprising (i) levodopa in an amount ranging from 50 mg to 300 mg, (ii) carbidopa in an amount ranging from 25 mg to 150 mg or a therapeutically equivalent amount of another aromatic amino acid decarboxylase inhibitor, and (iii) entacapone in an amount ranging from 50 mg to 300 mg, wherein the proportion of entacapone to carbidopa in said dosage form ranges from 0.3:1.0 to 3.2:1.0 by weight, a moderately potent COMT inhibitor in an amount ranging from 25 mg to 200 mg, wherein the proportion of said COMT inhibitor to carbidopa in said dosage form ranges from 0.16:1.0 to 3.08:1.0 by weight, or a highly potent COMT Inhibitor in an amount ranging from 1 mg to 100 mg, wherein the proportion of said COMT inhibitor to carbidopa in said dosage form ranges from 0.006:1.0 to 1.54:1.0 by weight. Pharmaceutical dosage forms used in said methods are also disclosed.

U.S. Pat. No. 10,851,109 is directed to compounds and pharmaceutically acceptable salts thereof for the treatment and/or prevention of neurodegenerative and/or mitochondrial diseases, such as Parkinson's disease and Leigh's disease. The compounds and pharmaceutically acceptable salts thereof are of the class of nitrogenous bases, for example, pyrimidines, purines, pteridines, and pharmaceutically acceptable salts thereof.

U.S. Pat. No. 10,851,066 relates to the compound 2-(3-tert-Butylphenyl)-4,6-difluoro-1H-benzo[d]imidazole and its use. The compound activates Ppargc1a and, as a consequence, is useful for treating neuroinflammation and for treating a variety of neurodegenerative diseases.

U.S. Pat. No. 10,849,953 relates to a food supplement to reduce symptoms of Parkinson's disease. The food supplements contain a mixture of garlic, almond, peanut, tomato, grape, clove, black eye pea turmeric, tea and velvet bean.

U.S. Pat. No. 10,844,061 relates to pharmaceutical compositions comprising compounds of Formula Q: ##STR00001## wherein W is N(H)—, or —N(CH.sub.3)-, and Y is —C(.dbd.O)— or —O—, in free base or pharmaceutically acceptable salt form, and methods of use in the treatment of diseases involving 5-HT.sub.2A receptor, serotonin transporter (SERT) pathway and/or the dopamine D.sub.2 receptor pathway, and methods of treating conditions of the central nervous system therewith.

U.S. Pat. No. 10,829,454 relates to the compounds of formula I, formula II and formula III or its pharmaceutical acceptable salts, as well as polymorphs, solvates, enantiomers, stereoisomers and hydrates thereof. The pharmaceutical compositions comprising an effective amount of compounds of formula I, formula II and formula III, and methods for treating or preventing Parkinson's disease may be formulated for oral, buccal, rectal, topical, transdermal, transmucosal, intravenous, parenteral administration, syrup, or injection. Such compositions may be used to treatment or management of Parkinson's disease such as Parkinson's disease, scleroderma, restless leg syndrome, hypertension and gestational hypertension.

U.S. Pat. No. 10,828,276 relates to embodiments of the present invention that are directed to the administration of bryostatins and bryostatins and retinoids for the treatment of disease responsive to increases in alpha secretase activity. Inventions of the present application are directed to the treatment of neuro-degenerative diseases such as Hutchinson Disease, Parkinson's disease, Down's syndrome and Alzheimer's disease and virus latency diseases such as HIV and Herpes, cancers such as prostate, melanomas, lymphomas and renal cancers, esophageal and opthalmic diseases such as glaucoma.

U.S. Pat. No. 10,813,979 provide methods, compositions, and kits for diagnosing or treating neurodegenerative or neuroinflammatory conditions. Also provided are methods for identifying modulators of neurodegenerative or neuroinflammatory conditions.

U.S. Pat. No. 10,793,515 provides a series of novel amides showing broad pharmaceutical activity. Compounds described herein are effective as anticonvulsants, chemical countermeasures, and analgesics. Such compounds also show, neuroprotective/neuroreparative effects and activity against spinal muscular atrophy. Such pharmaceutically active compounds show utility in the treatment of central nervous system (“CNS”) diseases and disorders, such as anxiety, depression, insomnia, migraine headaches, schizophrenia, neurodegenerative diseases (e.g., Parkinson's disease, Alzheimer's, ALS, and Huntington's disease) spasticity, and bipolar disorder. Furthermore, such compounds may additionally find utility as analgesics (e.g., for the treatment of chronic or neuropathic pain) and as neuroprotective agents useful in the treatment of stroke(s), and/or traumatic brain and/or spinal cord injuries.

U.S. Pat. No. 10,787,669 relates to compounds comprising oligonucleotides complementary to a Leucine-Rich-Repeat-Kinase (LRRK2) RNA transcript. Certain such compounds are useful for hybridizing to a LRRK2 RNA transcript, including but not limited to a LRRK2 RNA transcript in a cell. In certain embodiments, such hybridization results in modulation of splicing of the LRRK2 transcript. In certain embodiments, such compounds are used to treat one or more symptoms associated with Parkinson's disease.

U.S. Pat. No. 10,786,472 relates to a pharmaceutical gel composition for intra-intestinal administration that comprises (i) a dopamine replacement agent, (ii) a dopamine decarboxylase inhibitor (DDI), and (iii) a COMT inhibitor.

U.S. Pat. No. 10,583,125 is directed to a method for treating a neurodegenerative disease such as amyotrophic lateral sclerosis (ALS), Alzheimer disease, Parkinson's disease, Huntington's disease, frontotemporal degeneration, dementia with Lewy bodies, a motor neuron disease, or a demyelinating disease. The method comprises administering to a subject in need thereof a Ppargc1a activator 2-(4-tert-butylphenyl)-1H-benzimidazole, 2-[4-(1,1-dimethylethyl)phenyl]-1H-benzimidazole, in an effective amount. A preferred route of administration is oral administration.

U.S. Pat. No. 10,555,922 relates to a method of treating a dopamine related disorder in a subject, the method comprises the steps of administering therapy to a subject, the therapy comprising a plurality of doses of levodopa over a selected time period, in combination with a dopamine decarboxylase inhibitor (DDI) and a catechol-O-methyltransferase (COMT) inhibitor, wherein: (i) the dose of levodopa is lower; (ii) the dose of DDI is lower; (iii) the dosing of levodopa is less frequent; and/or (iv) the dosing of DDI is less frequent As compared with a reference regimen over the selected time period.

U.S. Pat. No. 10,538,553 disclosed compounds for the treatment of neurodegenerative diseases and compositions comprising the same.

U.S. Pat. No. 10,526,383 relates, in one aspect, to use of ghrelin splice variant or an analogue thereat: or a ghrelin splice variant-like compound for the preparation of a medicament for one or more of: treatment and/or prevention of neuronal damage and/or neurodegeneration and, prophylaxis or treatment of neuronal damage and/or neurodegenerative disease, Parkinson's disease, Alzheimer's disease, depression stimulation of neuronal activity. A further aspect relates to a number of new ghrelin splice variant like compounds and uses thereof, as well as to pharmaceutical compositions and medical packaging comprising the new ghrelin splice variant-like compounds.

U.S. Pat. No. 10,525,107 described methods and compositions for treating and/or preventing aging-related conditions. The compositions used in the methods include fractions derived from blood plasma with efficacy in treating and/or preventing aging-related conditions such as neurocognitive disorders.

U.S. Pat. No. 10,428,102 provides, in part, compounds for use as antiproliferative, chemotherapeutic, antiviral, cell sensitizing or adjuvant agents, and pharmaceutical compositions including the compounds. The compounds may be for use in treating diseases and disorders related to cell proliferation such as cancer, or in treating diseases and disorders which are linked to aberrant control of protein synthesis, such as cancer, viral infection, muscle wasting, autistic spectrum disorders, Alzheimer's disease, Huntingdon's disease and Parkinson's disease.

U.S. Pat. No. 10,421,724 provides novel compounds as well as compositions and methods using the same for preventing and/or treating degenerative disorders of the central nervous system. In particular, the present invention provides methods for preventing and/or treating Parkinson's disease.

U.S. Pat. No. 10,398,713 discloses methods of treating diseases and disorders with sulfasalazine and pharmaceutical formulations of sulfasalazine where the bioavailability of the sulfasalazine is increased. In another embodiment, the present application also provides dosing regimens for treating neurodegenerative diseases and disorders with compositions comprising sulfasalazine.

U.S. Pat. No. 10,391,067 provides prevention and treatment of neurodegenerative diseases through autophagy activity mediated by a synthetic ligand or arginylated BIP binding to the P62 ZZ domain.

U.S. Pat. No. 10,159,665 present embodiments of a method of inhibiting amyloid plaque deposition. In some embodiments, the method comprises: administering to an individual known to have a decreased expression level of Bri3 an amount of a nitroxide antioxidant effective to increase the BRI3 expression level relative to the decreased expression level, whereby the increased expression level of Bri3 inhibits amyloid plaque deposition via inhibition of amyloid precursor protein processing.

U.S. Pat. No. 9,782,602 A transcranial magnetic stimulation device in accordance with embodiments of the present invention comprises a head mount for disposition on a head of a patient and configured with a plurality of attachment points, a plurality of magnetic assembly devices connected to the plurality of attachment points, a given magnetic assembly device equipped with an actuator device to actuate a magnet, is addressable, and configured to receive a control signal addressed to the given magnetic assembly device, and a processor having a memory and configured by program code. The processor is configured to: select one or more treatment protocol units, generate a control signal using at least information contained in the selected treatment protocol units, energize at least one magnetic assembly device over a period of time to cause the magnet to actuate according to the control signal, and monitor the patient response to energizing to addressable actuator. The TMS method may be use wherein the treatment is selected from depression, neurological and psychiatric disorders, migraines, aphasia, anxiety, Parkinson's disease, tinnitus, autism, schizophrenia, Alzheimer's, ALS, stroke (e.g. ischemic), Myotonic Dystrophy type 1 (DM1), stuttering, epilepsy, Parkinson's disease, visceral pain and dystonia, cocaine, opioid and other addictive behavior.

U.S. Pat. No. 9,782,590 present a system for treating a patient that comprises a stimulator for stimulating brain tissue, a controller for setting stimulation parameters and a diagnostic tool for measuring patient parameters and producing diagnostic data. The stimulation parameters comprise test stimulation parameters and treatment stimulation parameters. The stimulator delivers test stimulation energy to the brain tissue based on at least one test stimulation parameter and delivers treatment stimulation energy to the brain tissue based on at least one treatment stimulation parameter. One or more treatment stimulator parameters are determined based on the diagnostic data produced by the diagnostic tool The system is constructed and arranged to treat a neurological disease or a neurological disorder. Methods of treating a neurological disease or neurological disorder are also provided.

U.S. Pat. No. 9,453,079 present a method of treating an individual having a neurological disease or disorder characterized by amyloid .beta. pathology, comprising administering to the individual an effective amount of an antibody or fragment thereof that binds to .beta.-site amyloid precursor protein cleaving enzyme 1 (BACE1), wherein the antibody or fragment thereof comprises a) an HVR-H1 comprising the amino acid sequence GFTFX.sub.13GYX.sub.14IH (SEQ ID NO:26), wherein X.sub.13=S or L and X.sub.14=A or G; b) an HVR-H2 comprising the amino acid sequence GWISPAGGSTDYADSVKG (SEQ ID NO: 24); c) an HVR-H3 comprising the amino acid sequence of GPFSPWVMDY (SEQ ID NO: 25), d) an HVR-L1 comprising the amino acid sequence of RASQX.sub.1VX.sub.2X.sub.3X.sub.4X.sub.5A (SEQ ID NO:17), wherein X.sub.1=D or V; X.sub.2=S or A; X.sub.3=T or N; X.sub.4=S or A; X.sub.5=V or L; e) an HVR-L2 comprising the amino acid sequence of X.sub.6ASFLYS (SEQ ID NO:18) wherein X.sub.6=S or L; and f) an HVR-L3 comprising the amino acid sequence of QQX.sub.7X.sub.8X.sub.9X.sub.10X.sub.11X.sub.12T (SEQ ID NO:19), wherein X.sub.7=S, F, G, D or Y; X.sub.8=Y, P, S, or A; X.sub.9=T or N; X.sub.10=T, Y, D or S; X.sub.11=P or L; X.sub.12=P or T; wherein the antibody reduces or inhibits the activity of BACE1. The method wherein the neurological disease or disorder is selected from the group consisting of Alzheimer's disease, stroke, traumatic brain injury, Lewy body disease, Parkinson's disease, and glaucoma.

U.S. Pat. No. 9,415,233 described herein methods of treating neurological injury and conditions, in particular neurological pain. These treatment methods can include the steps of generating a pulsed electromagnetic field from a pulsed electromagnetic field source and applying the pulsed electromagnetic field in proximity to a target region.

U.S. Pat. No. 9,364,501 features methods of treating a macrophage-associated neurodegenerative disease such as amyotrophic lateral sclerosis (ALS), Alzheimer's disease (AD), or multiple sclerosis (MS) in a subject by administering chlorite in an amount effective to decrease blood immune cell activation. The invention also features methods of monitoring therapy by assessing blood immune cell activation before and after therapy.

U.S. Pat. No. 9,327,118 present a method of treating a disease or a disorder in a patient, the method comprising: positioning one or more energy transmitters within a container comprising an electrically conducting fluid such that the one or more energy transmitters is at least partially surrounded by and in contact with the electrically conductive fluid, wherein the container is coupled to a power source, wherein the container and the power source are housed in a housing, wherein the housing comprises a surface; positioning the surface of the housing against an outer skin surface of a neck of a patient; and applying sufficient energy to the one or more energy transmitters via the power source to generate one or more electrical impulses at or near a vagus nerve of the patient, wherein the one or more electrical impulses is sufficient to stimulate an activity of the vagus nerve to treat the disease or the disorder.

U.S. Pat. No. 9,259,465 present a method of treating a central nervous system disease or disorder in a patient in need thereof, wherein said central nervous system disease or disorder is characterized by the presence of at least one abnormal protein, said method comprising administering to said patient: a) at least one immunogen capable of inducing a humoral immune response against said abnormal protein, and b) at least one adjuvant that stimulates regulatory T cells, wherein said central nervous disease or disorder is amyotrophic lateral sclerosis (ALS) and said immunogen is superoxide dismutase (SOD).

U.S. Pat. No. 8,900,284 relates to an implantable device that delivers an effective amounts of red light to the substantia nigra as a treatment for Parkinson's Disease (PD). The method of treating a patient having a neurodegenerative disease, comprising the steps of: a) implanting a device comprising an antenna and a light emitting diode (LED) in a brain of the patient, b) transcutaneously transmitting Rf energy to the antenna in an amount sufficient to power the LED, and c) irradiating a patient's brain with an effective amount of red or infrared light from the powered LED, wherein irradiation of the brain is carried out with between about 0.2 J/cm.sup.2 and 50 J/cm.sup.2 energy and for a duration on the order of minutes, wherein irradiation is carried out at an intensity of between ⅓ mW/cm2 and 625 mW/cm.sup.2.

U.S. Pat. No. 8,473,060 present a treatment for Parkinson's Disease that uses a stimulus electrode implanted in a subthalamic nucleus with a chemosensor implanted in a globus pallidus pars interna (GPi) of the subject. A level of a neurochemical is sensed with the chemosensor, and compared to a desired level. When the level of the neurochemical is less than desired, an electrical stimulation is provided to the stimulus electrode. In alternative embodiments, the neurochemical sensed is glutamate or dopamine. An alternative system uses a chemosensor implanted in the striatum instead of the GPi. An alternative system for treating benign essential tremor uses a stimulus electrode implanted in the thalamus with feedback taken from a chemosensor in the striatum.

U.S. Pat. No. 8,222,300 provides methods of using halogenated volatile compounds, e.g., halogenated ether, for treating a neurological disorder, e.g., Parkinson's disease. The method of treating Parkinson's disease in a subject in need thereof comprising administering a therapeutically effective amount of a halogenated volatile compound to the subject, such that the Parkinson's disease is treated or, wherein the halogenated volatile compound is selected from the group consisting of a halogenated ether, a halogenated alkane and a halogenated benzene. The method also includes using the halogenated volatile compound administered in combination with oxygen and/or carbon dioxide

U.S. Pat. No. 8,167,921 present a method for treating a subject having Parkinson's disease is provided. The method includes noninvasively delivering light energy having a wavelength of about 630 nanometers to about 904 nanometers to the brain of the subject. Delivering the light energy can include (i) irradiating the scalp with light energy having an incident power density less than 1000 mW/cm.sup.2 and (ii) transmitting a portion of the light energy through the scalp and the skull to the brain.

U.S. Pat. No. 7,875,273 present cells derived from postpartum tissue such as the umbilical cord and placenta, pharmaceutical compositions comprising such cells, and methods for using such cells and pharmaceutical compositions to treat patients having a neurodegenerative condition of the substantia nigra or striatum, such as Parkinson's disease.

U.S. Pat. No. 7,105,183 features methods of treating a macrophage-associated neurodegenerative disease such as amyotrophic lateral sclerosis (ALS), Alzheimer's disease (AD), or multiple sclerosis (MS) in a subject by administering chlorite in an amount effective to decrease blood immune cell activation. The invention also features methods of monitoring therapy by assessing blood immune cell activation before and after therapy.

There are not any patent or patent application regarding a pulse oxygen therapy for treating neurodegenerative disorders such as Parkinson Disease (PD), Alzheimer's Disease (AD) and other dementias, amyotrophic lateral sclerosis (ALS), and Lymphedema, arthritis and depression among others.

### SUMMARY OF THE INVENTION

A novel oxygen pulse therapy methods of treating subjects suffering from neurodegenerative disorders such as Parkinson Disease (PD), Alzheimer's Disease (AD) and other dementias, amyotrophic lateral sclerosis (ALS), and Lymphedema, arthritis and depression among others is provided. Aspects of the methods including administering to the subjects an effective amount of oxygen through the respiratory tract are included.

### DESCRIPTION OF THE INVENTION

### Systems of the Human Body

### Circulatory System

The circulatory system consists of the heart and blood vessels (arteries, veins and capillaries). The heart propels the circulation of the blood, which serves as a “transportation system” to transfer oxygen, fuel, nutrients, waste products, immune cells and signaling molecules (i.e. hormones) from one part of the body to another. Paths of blood circulation within the human body can be divided into two circuits: the pulmonary circuit, which pumps blood to the lungs to receive oxygen and leave carbon dioxide, and the systemic circuit, which carries blood from the heart off to the rest of the body. The blood consists of fluid that carries cells in the circulation, including some that move from tissue to blood vessels and back, as well as the spleen and bone marrow.

### Digestive System

The digestive system consists of the mouth including the tongue and teeth, esophagus, stomach, (gastrointestinal tract, small and large intestines, and rectum), as well as the liver, pancreas, gallbladder, and salivary glands. It converts food into small, nutritional, non-toxic molecules for distribution and absorption into the body. These molecules take the form of proteins (which are broken down into amino acids), fats, vitamins and minerals (the last of which are mainly ionic rather than molecular). After being swallowed, food moves through the gastrointestinal tract by means of peristalsis: the systematic expansion and contraction of muscles to push food from one area to the next.

### Endocrine System

The endocrine system consists of the principal endocrine glands:

the pituitary, thyroid, adrenals, pancreas, parathyroids, and gonads, but nearly all organs and tissues produce specific endocrine hormones as well. The endocrine hormones serve as signals from one body system to another regarding an enormous array of conditions, and resulting in variety of changes of function.

### Immune System

The immune system consists of the white blood ceils, the thymus, lymph nodes and lymph channels, which are also part of the lymphatic system. The immune system provides a mechanism for the body to distinguish its own cells and tissues from outside cells and substances and to neutralize or destroy the latter by using specialized proteins such as antibodies, cytokines, and toil-like receptors, among many others

### Integumentary System

The integumentary system consists of the covering of the body (the skin), including hair and nails as well as other functionally important structures such as the sweat glands and sebaceous glands. The skin provides containment, structure, and protection for other organs, and serves as a major sensory interface with the outside world.

### Lymphatic System

The lymphatic system extracts, transports and metabolizes lymph, the fluid found in between cells. The lymphatic system is similar to the circulatory system in terms of both its structure and its most basic function, to earn, a body fluid

### Musculoskeletal System

The musculoskeletal system consists of the human skeleton (which includes bones, ligaments, tendons, and cartilage) and attached muscles. It gives the body basic structure and the ability for movement. In addition to their structural role, the larger bones in the body contain bone marrow, the site of production of blood cells. Also, all bones are major storage sites for calcium and phosphate. This system can be split up into the muscular system and the skeletal system

### Nervous System

The nervous system consists of the body's neurons and glial cells, which together form the nerves, ganglia and gray matter which in turn form the brain and related structures. The brain is the organ of thought, emotion, memory, and sensory processing; it serves many aspects of communication and controls various systems and functions. The special senses consist of vision, hearing, taste, and smell. The eyes, ears, tongue, and nose gather information about the body's environment.

From a structural perspective, the nervous system is typically subdivided into two component parts: the central nervous system (CNS), composed of the brain and the spinal cord; and the peripheral nervous system (PNS), composed of the nerves and ganglia outside the brain and spinal cord. The CNS is mostly responsible for organizing motion, processing sensory information, thought, memory, cognition and other such functions. It remains a matter of some debate whether the CNS directly gives rise to consciousness. The peripheral nervous system (PNS) is mostly responsible for gathering information with sensory neurons and directing body movements with motor neurons.

From a functional perspective, the nervous system is again typically divided into two component parts: the somatic nervous system (SNS) and the autonomic nervous system (ANS). The SNS is involved in voluntary functions like speaking and sensory processes. The ANS is involved in involuntary processes, such as digestion and regulating blood pressure

The nervous system is subject to many different diseases. In epilepsy, abnormal electrical activity in the brain can cause seizures. In multiple sclerosis, the immune system attacks the nerve linings, damaging the nerves' ability to transmit signals. Amyotrophic lateral sclerosis (ALS), also known as Lou Gehrig's disease, is a motor neuron disease which gradually reduces movement in patients. There are also many other diseases of the nervous system.

### Reproductive System

The primary purpose of the reproductive system is to produce new humans in the form of children and ensure their sexual development so that they can produce new humans too. It is composed of the gonads (testes and ovaries) which produce eggs and sperm cells (gametes) and produce hormones necessary for proper sexual development. The rest of the glands and ducts of the reproductive system are responsible for the transportation and sustaining of the gametes and to nurture the offspring.

### Respiratory System

The respiratory system consists of the nose, nasopharynx, trachea, and lungs. It brings oxygen from the air and excretes carbon dioxide and water back into the air. First, air is pulled through the trachea into the lungs by the diaphragm pushing down, which creates a vacuum. Air is briefly stored inside small sacs known as alveoli before being expelled from the lungs when the diaphragm contracts again. Each alveolus is surrounded by capillaries carrying deoxygenated blood, which absorbs oxygen out of the air and into the bloodstream

### Urinary System

The armory system consists of the two kidneys, two ureters, bladder, and urethra. It removes waste materials from the blood through urine, which carries a variety of waste molecules and excess ions and water out of the body.

### Brain Memory of Body Functions

The brain memory of body functions corresponds to all the information located in the brain that control the normal functioning of all system of the whole organism. This memory is hereditary, evolutionary and self-improve and autopoyetic. Most of the diseases and failures of the body is due to malfunctioning of this memory and the sanitation must be done through the resetting of his complex input and outputs signals and procedures.

The brain memory of the body functions is composed of the following components:

### Immune Memory

It is where all diseases and foreign microorganism attacks are controlled. Then control all the biochemical reaction linked to pathological microorganism action, all biochemical signal's given by a microorganism attacks, the immunological answer as command lymphocytes production in time and marrow.

### Physiological Memory

Where all human organism systems are controlled.

### Emotional Memory

Where all the human affective processes are controlled

### Cognitive Memory

Where all the human knowledge and processing are controlled

Origin of diseases and failures of the human body.

We postulate that all the diseases and failures of the human body are caused, except of those caused by physical loss of some organ and tissues, by a malfunction or failure of the brain memory of the body functions which control the production of chemical (sensors, transmitters and others) and production of essential organism materials (cells, blood production, lymphocyte's production, hormone production and others.

Then our approach for treating any disease it is to restore the brain memory of the body functions.

### How to Restore the Brain Memory of the Body Functions?

Our approach is for restoring the brain memory of the body function we need to discover some procedure that can act as a resetting of such a memory.

### What is Our Discovery?

We discover that treating the organism with oxygen pulses through the respiratory tract we can cure most of the diseases. Then the oxygen pulses reset and restore the brain memory of the body functions permitting the elimination of most of the diseases.

Our practical studies using oxygen pulses eliminate diseases that are caused by failures of the physiological memory, emotional memory and cognitive memory (oxygen pulses applied to treat Parkinson disease, Alzheimer's disease, amyotrophic lateral sclerosis, Limphoedema, arthritis and depression).

Evidence that Parkinson's disease (PD) may start off in the gut is mounting, according to new research showing proteins thought to play a key role in the disease can spread from the gastrointestinal tract to the brain.

The human body naturally forms a protein called alpha-synuclein which is found, among other places, in the brain in the endings of nerve cells. However, misfolded forms of this protein that clump together are linked to damage to nerve cells, a deterioration of the dopamine system and the development of problems with movement and speech—hallmarks of Parkinson's disease. The latest findings, which are based on studies in mice, back up a long-held theory that abnormally folded alpha-synuclein may start off in the gut and then spread to the brain via the vagus nerve a bundle of fibers that starts in the brainstem and transports signals to and from many of the body's organs, including the gut.

ALS or MND is characterized by gradual degeneration of motor neuron cells in the spinal cord and brain, which ultimately leads to progressive weakness and paralysis of muscle and death. ALS occurs in two clinically indistinguishable forms, referred to as a sporadic form and a familial form. The pathogenesis of ALS is incompletely understood, although different hypotheses have been suggested, including mitochondria dysfunction, mutation in the superoxide dismutase gene, and defects in neuronal glutamate transport. Autoimmunity has also been hypothesized to be involved in ALS pathogenesis (Appel et al. 1993). In addition, several recent studies have suggested that the immune system may be actively involved in the disease process of ALS, with observations of activated microglia, IgG deposits, increased FcR expression, and dysregulation of cytokine expression in the spinal cord of ALS patients (Troost et al. 1989, Engelthardt et al. 1990, Schiffer et al. 1996, Hayashi et al. 2001).

Recent clinical and pathological studies have shown that involvement outside the motor neuron system is relatively common in Amyotrophic Lateral Sclerosis (ALS) (Hayashi et al. 2001, Obal et al. 2001, Sola et al. 2002, Ono et al. 2001, Alexianu et al. 2001). Microglia/macrophage activation and inflammatory response have been implicated in ALS disease progression (Obal et al. 2001, McGeer et al. 2002). However, few studies to date have explored the status of the systemic immune response in ALS. Despite intensive investigation, ALS has no known cause or effective therapy.

Retroviral infection has recently been implicated in the pathogenesis of an ALS-like syndrome in patients with HIV-associated disease. Moulignier et al., 2001 reported the outcome of six HIV-1-infected patients with a neurologic disorder mimicking ALS and all those patients stabilized or improved with antiretroviral therapy. MacGrowen et al. 2001, also reported a dramatic clinical response to antiretroviral therapy in an ALS-like syndrome with new HIV infection.

Approximately one-quarter of individuals with AIDS develop neuropathological symptoms. Infection by HIV-1 causes inflammation within the brain and neuronal degeneration (Power et al. 2001) resulting in HIV-associated dementia (HAD) or the less severe minor cognitive and motor disorders (Janssen et al. 1991, McArthur et al. 1993).

The mechanisms underlying HIV-associated neuronal injury are incompletely understood. Various studies have suggested that monocytes/macrophage activation may play a significant role in the pathogenesis of many neurological diseases (Smits et al. 2000 Eur. Fiala et al. 2002, Minagar et al. 2002), including HIV-associated neurologic disorders (Pulliam et al. 1997, Diesing et al. 2002). Indeed, the best pathological correlate for HIV-associated neurologic disorders, especially HAD, is the number of activated mononuclear phagocytes (perivascular and parenchymal blood-derived macrophages and microglia), not the absolute levels of virus in brain per se (Glass et al. 1995, Adamson et al. 1999). Similar findings have been reported for AIDS related encephalopathy (SIVE) (Williams et al. 2002). Macrophage activation has been reported in spinal cords of patients with ALS disease (Appel et al. 1993, Engelthardt et al. 1990, Obal et al. 2001, McGeer et al. 2002) although the role of macrophage activation in ALS pathogenesis has not been previously determined.

Studies on blood from patients with HAD (Liu et al. 2000) and monkeys with SIVE (Williams et al. 2002) have shown a direct relationship between the presence of activated blood macrophages and central nervous system (CNS) disease. These activated macrophages are thought to mediate blood brain harrier (BBB) breakdown and directly contribute to CNS pathogenesis.

Alzheimer's disease (AD) is the most common form of dementia among the elderly. Various studies have suggested that macrophage activation may be involved in AD (see, e.g., WO 99/21542). Currently the only definite way to diagnose AD is by post-mortem autopsy to assess the presence of amyloid plaques and tangles in brain tissue. Thus, AD diagnosis is generally a diagnosis of “possible” or “probable” AD. At specialized centers, doctors can diagnose AD correctly up to 90 percent of the time. Several tools are used to diagnose “probable” AD, including medical history, analysis of blood urine, or spinal fluid, to rule out other causes (e.g., thyroid deficiencies, infectious disease, etc.), brain scans, and neuropsychological tests to assess memory, problem solving, attention, counting, and language.

Multiple sclerosis (MS) is a chronic disease that is characterized by “attacks,” during which areas of white matter of the central nervous system, known as plaques, become inflamed. Inflammation of these areas of plaque is followed by destruction of myelin, the fatty substance that forms a sheath or covering that insulates nerve cell fibers in the brain and spinal cord. Myelin facilitates the smooth, high-speed transmission of electrochemical messages between the brain, spinal cord, and the rest of the body. Damage to the myelin sheath can slow or completely block the transmission of these electrochemical messages, which can result in diminished or lost bodily function. Multiple sclerosis (MS) is a condition that can affect the brain and spinal cord, causing a wide range of potential symptoms, including problems with vision, arm or leg movement, sensation or balance. It's a lifelong condition that can sometimes cause serious disability, although it can occasionally be mild. In many cases, it's possible to treat symptoms. Average life expectancy is slightly reduced for people with MS. It's most commonly diagnosed in people in their 20s and 30s, although it can develop at any age. It's about 2 to 3 times more common in women than men. MS is 1 of the most common causes of disability in younger adults.

Arthritis is the swelling and tenderness of one or more of your joints. The main symptoms of arthritis are joint pain and stiffness, which typically worsen with age. The most common types of arthritis are osteoarthritis and rheumatoid arthritis.

Osteoarthritis causes cartilage—the hard, slippery tissue that covers the ends of bones where they form a joint—to break down. Rheumatoid arthritis is a disease in which the immune system attacks the joints, beginning with the lining of joints.

Uric acid crystals, which form when there's too much uric acid in your blood, can cause gout. Infections or underlying disease, such as psoriasis or lupus, can cause other types of arthritis.

Treatments vary depending on the type of arthritis. The main goals of arthritis treatments are to reduce symptoms and improve quality of life.

Lymphedema, also known as lymphoedema and lymphatic edema, is a condition of localized swelling caused by a compromised lymphatic system. The lymphatic system functions as a critical portion of the body's immune system and returns interstitial fluid to the bloodstream. Lymphedema is most frequently a complication of cancer treatment or parasitic infections, but it can also be seen in a number of genetic disorders. Though incurable and progressive, a number of treatments can improve symptoms. Tissues with lymphedema are at high risk of infection because the lymphatic system has been compromised.

While there is no cure, treatment may improve outcomes. This commonly include compression therapy, good skin care, exercise, and manual lymphatic drainage (MLD), which together is known as combined decongestive therapy. Diuretics are not useful. Surgery is generally only used in those who are not improved with other measures.

The most common manifestation of lymphedema is soft tissue swelling, edema. As the disorder progresses, worsening edema and skin changes including discoloration, verrucous (wart-like) hyperplasia, hyperkeratosis, papillomatosis, dermal thickening and ulcers may be seen. Additionally, there is increased risk of infection of the skin, known as cellulitis.

Depression is a common illness worldwide, with more than 264 million people affected. Depression is different from usual mood fluctuations and short-lived emotional responses to challenges in everyday life. Especially when long-lasting and with moderate or severe intensity, depression may become a serious health condition. It can cause the affected person to suffer greatly and function poorly at work, at school and in the family. At its worst, depression can lead to suicide. Close to 800 000 people die due to suicide every year. Suicide is the second leading cause of death in 15-29-year-olds.

This disorder involves repeated depressive episodes. During these episodes, the person experiences depressed mood, loss of interest and enjoyment, and reduced energy leading to diminished activity for at least two weeks. Many people with depression also suffer from anxiety symptoms, disturbed sleep and appetite, and may have feelings of guilt or low self-worth, poor concentration and even symptoms that cannot be explained by a medical diagnosis.

Depending on the number and severity of symptoms, a depressive episode can be categorized as mild, moderate or severe. An individual with a mild depressive episode will have some difficulty in continuing with ordinary work and social activities but will probably not cease to function completely. During a severe depressive episode, it is unlikely that the sufferer will be able to continue with social, work or domestic activities, except to a limited extent. There are effective psychological and pharmacological treatments for moderate and severe depression.

All neurodegenerative disease has following main reasons (J. O. Hernandez, 2021):

1. Failure of the immune memory at the brain level to command production of proteins, antibodies, macrophages and neurotransmitters for avoiding death of neurons. Deficit of production of dopamine as in the case of PD.

2. Immune depression due to deficit of production of antibodies caused for the preexistence of other diseases (Arterial hypertension, diabetes, obesity and others) and causing the accumulation of beta amyloids as occurs in the case of AD.

3. Failure of the immune memory at the brain level to command production of myelin the fatty substance that forms a sheath or covering that insulates nerve cell fibers in the brain and spinal cord. Myelin facilitates the smooth, high-speed transmission of electrochemical messages between the brain, spinal cord, and the rest of the body. Damage to the myelin sheath can slow or completely block the transmission of these electrochemical messages, which can result in diminished or lost bodily function as in the case of MS.

4. Failure of the immune memory at the brain level to command the control of the production of antibodies and eventually producing autoimmunity as in ALS.

5. Failure of the brain physiological memory for command the production of proteins and avoiding degeneration of motor neurons cells in the spinal cord and brain leading to progressive weakness and paralysis of muscles and death such in ALS.

6. Failure of the brain physiological memory in the control of the lymphatic system after a cancer chirurgic treatment and in such a way producing lymphedema.

7. Failure of the brain physiological memory, to produce specific levels of some brain chemicals or chemical messengers (as neurotransmitters), control of nerve cell connections, nerve cell growth, and the functioning of nerve circuits.

8. Failure of the emotional brain memory for controlling the production of specific levels of some brain chemicals or chemical messengers (as neurotransmitters) and therefore producing depression.

We have discovered that administering pulses of oxygen through the respiratory tract serves to treat several neurodegenerative diseases and other neurological disorders such as Parkinson Disease (PD), Alzheimer's Disease (AD) and other dementias, ALS, multiple sclerosis (MS) and Limphoedema, arthritis and depression. Pulses of a reagent is a common tool in science and engineering used for study systems in an input-output basis for characterizing such systems. In our case is used for restart the brain memory to his normal functioning. Oxygen pulses act as a resetting button.

In the case of Parkinson's disease oxygen pulses stimulate the brain physiological memory for revitalizing and regenerating clusters of neurons in the midbrain and in such a way increasing dopamine and others neurotransmitters causing permanent cure of the disease.

Around 4,000 million years ago life began on earth surface. Firstly, as single cells organism and then more complexes ones. With the evolution of more complex organisms, start more complex mechanisms and processes of adaptation to the presence of single cell organism. That origin the initial immune system which permitted survival. This immune system was genetically transmitted and modified through the times. The immune system through the immune memory command the production of lymphocytes.

Oxygen is key element in the normal functioning of the human immunological system, additionally to several other functions in the human body. Oxygen reactions with glucose and protein in the presence of lymphocytes for producing antibodies. Additionally, oxygen reacts with other chemicals to produce neurochemicals essentials for the normal functioning of the organism.

Then hominid had an immune system commanded by the brain immune memory that permitted the survival of the specie, until the Homo sapiens appears and with increasing populations and social organization of them and unnatural conditions of life gave origin to sickness. In modern times growing urbanization and unhealthy environment and nutrition in the towns gave origin to several neurodegenerative diseases. However, due to the existence of an evolutionary immune memory that is located in the brain, these diseases were controlled. This memory contains all the information regarding microorganism and virus's infections and immune system answers and is continuously refreshed and finally is genetically transmitted.

The main basis of this invention are:

Oxygen therapy to treat neurodegenerative and other diseases.

At sea level, (non humidified) room air contains 21% O₂ which translates into an oxygen partial pressure of 160 mm Hg [0.21*760 mm Hg)]. However, the body mean tissue oxygen levels are much lower than this level. Alveolar air contains 14% oxygen (106 mm Hg), arterial oxygen concentration is 12% (91 mm Hg), venous oxygen levels are 5.3% (40 mm Hg), and mean tissue intracellular oxygen concentration is only 3% (22 mm Hg (Guyton, and Hall, 1996). Furthermore, direct microelectrode measurements of tissue O₂ reveal that parts of the brain normally experience O₂ levels considerably lower than total body mean tissue oxygen levels, reflecting the high oxygen utilization in brain. These studies also highlight considerable regional variation in average brain oxygen levels (Table 1) that have been attributed to local differences in capillary density. Mean brain tissue oxygen concentration in adult rates is 1.5% (Silver and Erecinska, 1988), and mean fetal sheep brain oxygen tension has also been estimated at 1.6% (Koos and Power, 1987).

TABLE 1
Regional rat brain tissue partial pressures of
oxygen measured by microelectrode

| Brain area | % O₂ |
|---|---|
| Cortex (gray) | 2.5-5.3 |
| Cortex (white) | 0.8-2.1 |
| Hypothalamus | 1.4-2.1 |
| Hippocampus | 2.6-3.9 |
| Pons, fornix | 0.1-0.4 |

“Physiologic” oxygen levels are the range of oxygen levels normally found in healthy tissues and organs. These levels vary depending on tissue type (Table 1). However, it is of note that this rate is below 15% in all tissues and below 8% in most tissues. Thus the physiological oxygen levels can range from about 15% to about 1.5% depending upon the region of the body being measured.

Diffusion of oxygen to the body tissues is possible due a pressure gradient of oxygen from the air till the mitochondria (Ganong W., 1999).

When oxygen diffuses through the capillary alveolus, 97% reacts with the hemoglobin and 3% becomes absorbed in the plasma. (Guyton A., Hall J., 1996).

If we inspire air at normal pressure of 760 mm Hg (O₂21%) alveolar oxygen content will be 14% (106 mm Hg) and arterial oxygen content 12% (91 mm Hg). However arterial oxygen content includes oxygen content in the Hemoglobin plus oxygen absorbed in the plasma. (Mathews and Van Holde, 2002)

Oxygen content in the Hemoglobin (ml/dL)=Hb (g/dL)*% Sat.*1.36 mg/dL

At 100 mmHg oxygen pressure and 100% Hemoglobin saturation Oxygen content in the arterial blood is 20.7 ml/dL

If we consider a normal cardiac flow of 1 l/min, oxygen transported to the tissues would be 20.7 l Oxygen/min or 0.924 mol-g of oxygen (5.57*10²³ molecules of oxygen/min.).

If we inspire oxygen enriched air of 31% O₂, arterial pressure of oxygen would be (31/21)*100=147 mm Hg

Also oxygen content at the brain tissue will be:

TABLE 2
Oxygen at the brain level with 31% oxygen inspired

| Brain area | % O₂ |
|---|---|
| Cortex (gray) | 3.69-7.79 |
| Cortex (white) | 1.17-3.0 |
| Hypothalamus | 2.0-3.0 |
| Hippocampus | 3.8-5.7 |
| Pons, fornix | 0.14-0.58 |

Oxygen content in the arterial blood=Oxygen in Hemoglobin+Oxygen absorbed in the plasma

Oxygen content in the arterial blood=20.4 g/dL+0.0031 ml/mm Hg/dL*147 mm Hg=20.85 ml/dL

If we consider a normal cardiac flow of 1 l/min, oxygen transported to the tissues would be 20.85 l Oxygen/min. This is an additional flow of oxygen of 0.15 l/min or (0.15/22.4)=0.006 mol-g of oxygen/min or 0.036*10²³ molecules of oxygen/min. This is an increase of 0.72% of the oxygen transport to the tissues.

If we inspire oxygen of 95% O₂, arterial pressure of oxygen would be (95/21)*100=452 mm Hg

TABLE 3
Oxygen at the brain level with 95 % oxygen inspired

| Brain area | % O₂ |
|---|---|
| Cortex (gray) | 11.3-23.8 |
| Cortex (white) | 3.58-9.18 |
| Hypothalamus | 6.12-9.18 |
| Hippocampus | 11.6-17.4 |
| Pons, fornix | 0.42-1.77 |

Then increase of the oxygen reaching the brain tissue has a deep impact and permit the resetting of all the brain memory functions, causing the cure of the neurodegenerative diseases.

Oxygen content in the arterial blood=Oxygen in Hemoglobin+Oxygen absorbed in the plasma

Oxygen content in the arterial blood=20.4 g/dL+0.0031 ml/mm Hg/dL*452 mm Hg=21.80 ml/dL

If we consider a normal cardiac flow of 1 l/min, oxygen transported to the tissues would be 21.80 l Oxygen/min. This is an additional flow of oxygen of 1.4 l/min or (1.40/22.4)=0.0625 mole-g of oxygen/min or 0.376*10²³ molecules of oxygen/min. This is an increase of 6.76% of oxygen flow to the tissues.

In term of molecules of oxygen per minute is a huge increase.

When the air circulate through the respiratory tract becomes warmed and humid and due to the water vapor pressure at alveolar level the PO₂ decrease approximately to 110 mm Hg. Then cause to the PCO₂ and the pressure loss of the diffusion through the capillary alveolus decreases to 100 mm Hg, and arrives to 95 mm Hg at the left atrium due to the anatomic short circuit. Pressure oxygen arterial blood transported to the tissues goes to 90 mm Hg and to 40 mm Hg in the capillaries. At the cellular membrane level goes to 10 mm Hg and in the mitochondria oscillate between 1 and 5 mm. (Mendez and Zeledon F, 2004).

A theory of neurodegenerative diseases (Hernandez J. O. 2021).

In the case of Parkinson's disease oxygen pulses stimulate the brain physiological memory for revitalizing and regenerating clusters of neurons in the midbrain and in such a way increasing dopamine and others neurotransmitters causing permanent cure of the disease.

In the case of AD, antibodies neutralize beta amyloids avoiding blocking of neurons. The whole process develops commanded by the brain immune memory following the next simplified mechanism, similar to a series kinetic reaction scheme:

I Beta amyloids production→II Antibodies production→III beta amyloids elimination

A simplified series kinetics reaction rate can be imagined for beta amyloid elimination:
I. Beta amyloids production. In 1986 was discovered the first Aizheimer's disease gene, known as APP, which provides instructions for making amyloid protein precursor (APP (Tanzi el al. (986). When this protein is cut (or cleaved) by enzymes—first, beta secretase, followed by gamma secretase—the byproduct is amyloid beta (sometimes shortened to Abeta) Later in 2013 several researchers (Tanzi el al. 2013), showed that a form of APP that has undergone a process called palmitoylation (pal APP) gives rise to amyloid beta. That study indicated that, within the neuron, palAPP is transported in a fatty vesicle (or sac) known as a lipid raft. palAPP is stabilized and prepared for cleavage by beta secretase in special lipid rafts within the neuron known as mitochondria-associated endoplasmic reticulum membranes (MAMs). For the first time not only was showed that the MAM is where pal APP is processed by beta secretase to make Abeta, but that this happens exclusively in axons and neuronal processes where Abeta does most of its damage. preventing assembly of MAMs, either with gene therapy or a drug that blocked a key protein called the sigma-1 receptor (SLR) dramatically decreased beta secretase cleavage of pal APP in axons and lowered Abeta production. Conversely, a drug that activated SIR triggered an increase in beta secretase cleavage of palAPP and increased production of amyloid beta in axons. (Tanzi el Al. 2021)
II. Antibodies production (oxygen+proteins+glucose+Mg→antibodies)
III. Beta amyloids+antibodies→beta amyloids elimination.

In this kinetic scheme for the step I, naming the reaction rates as R₁, R₂, R₃ and R₄, the slower reaction rate controls the global rate of the process. Our assumption is that reaction 4 is the slower one.

The step II occurs as follow:

II Antibodies production
6. Oxygen diffusion through pulmonary alveolus→
7. Oxygen absorption in blood giving absorbed oxygen→
8. Oxygen diffusion through red blood cell membrane→
9. Oxygen reaction with Hemoglobin (Hb) in the red blood cell to produce Hb-O₂ (active oxygen)→
10. Immune memory command the production of lymphocytes (mainly T and B) by thymus gland and bone marrow→
11. Reaction of Hb-O₂ (active oxygen) with proteins and glucose mediates by lymphocytes (T+B) and Mg ions to produce antibodies.

In this kinetic scheme for the step II, naming the reaction rates as R₆, R₇, R₈, R₉, R₁₀ and R₁₁ the slower reaction rate that controls the global rate of the process would be the reaction 11.

Under a normal sane organism immune system signal a neutralizing of the beta amyloids occurs under the neutralization step III following the reaction scheme:

### III Beta Amyloids Neutralization

12. beta amyloids produced at the neuron synapsis+antibodies presents at the neuron synapsis→beta amyloids neutralization or destruction (Reaction rates R₁₂)

Our approach is that the innate immune and chemical messenger synthesis memory located in the brain, which is evolutionary and hereditary, work properly no neurodegenerative diseases prosper and the organism is healthy. This memory is not linked to any resident immune cells of the central nervous system CNS, such as Microglial cells (MC), and as being evolutionary and hereditary is located in many neurons linked in such a way to ensure the homeostasis of synapses and the communication with organs working in the production of lymphocytes T and B for generating antibodies and rejecting beta amyloids as mentioned in the kinetic scheme proposed.

### Pulse Oxygen Therapy.

In our pulse oxygen therapy in an immune depress organism alerts and wakes the immune system memory for producing lymphocytes T and B and enhancing the reaction rate R₁₁ which permit finally by reaction 12 the microorganism neutralization. The oxygen pulses act a resetting button of normal functioning of the brain memory of the body functions.

An oxygen pulse to the lungs of a neurodegenerative disease affected person would have following consequences:
1. To increase the mass transfer of oxygen to the blood to normal levels and consequentially increasing antibodies production.
2. To restart the immune memory for increasing the signal for production of more lymphocytes T and B and therefore increasing the production of antibodies
3. To restart the physiological brain memory for revitalizing and regenerating clusters of neurons in the midbrain and in such a way increasing dopamine and others neurotransmitters causing permanent cure of PD
4. Restart of the immune memory functions at the brain level to command the control of the production of antibodies and eventually producing autoimmunity as in ALS.
5. Restart of the brain physiological memory for command the production of proteins and avoiding degeneration of motor neurons cells in the spinal cord and brain leading to progressive weakness and paralysis of muscles and death such in ALS.
6. Restart the brain physiological memory to command production of myelin the fatty substance that forms a sheath or covering that insulates nerve cell fibers in the brain and spinal cord. Myelin facilitates the smooth, high-speed transmission of electrochemical messages between the brain, spinal cord, and the rest of the body. Damage to the myelin sheath can slow or completely block the transmission of these electrochemical messages, which can result in diminished or lost bodily function as in the case of MS.
7. Restart of the brain physiological memory in the control of the lymphatic system after a cancer chirurgic treatment and in such a way avoiding lymphedema.
8. Restart of the brain physiological memory to produce specific levels of some brain chemicals or chemical messengers (as neurotransmitters), control of nerve cell connections, nerve cell growth, and the functioning of nerve circuits and then controlling arthritis.
9. Restart of the emotional brain memory for controlling the production of specific levels of some brain chemicals or chemical messengers (as neurotransmitters) and therefore controlling depression

Our pulse oxygen therapy is directed to produce at the brain level a response for producing lymphocytes, increasing the production of antibodies and finally neutralizing the beta amyloids neurons blocking.

Also if we increased the concentration of oxygen at the lung, for instance to 31% or larger, then we are restoring or increasing the efficiency of oxygen mass transfer to the blood. The brain then received this oxygen pulse and sent an order to the organs responsible (thymus and bone marrow) for producing lymphocytes T and B, enhancing its production and consequentially increasing the rate of reaction 12 and neutralizing the beta amyloids agglomeration.

In the case of Parkinson's disease oxygen pulses stimulate the brain physiological memory for revitalizing and regenerating clusters of neurons in the midbrain and in such a way increasing dopamine and others neurotransmitters causing permanent cure of the disease.

Restore of the physiological memory functions at the brain level to command production of myelin the fatty substance that forms a sheath or covering that insulates nerve cell fibers in the brain and spinal cord. Myelin facilitates the smooth, high-speed transmission of electrochemical messages between the brain, spinal cord, and the rest of the body. Damage to the myelin sheath can slow or completely block the transmission of these electrochemical messages, which can result in diminished or lost bodily function as in the case of MS.

The oxygen pulses to the brain increasing the oxygen rate transported to the blood and awakening and restarting the immune brain memory, physiological brain memory, emotional brain memory is realized using following alternative procedures:
1. Enriched air (31% O₂) pulses at 6 liters/minute during say 60 minutes through Venturi oxygen masks
2. Oxygen (90-99% O₂) pulses at 6 liters/minute during say 15 minutes through oxygen mask with reservoir no rebreathing.

In our therapy the oxygen pulses are administered in consonance with the advance of the neurodegenerative diseases (PD, AD, ALS, MS, Arthritis, Lymphedema and Depression) as described as follow:
a. At the beginning of the neurodegenerative disease with the apparition of the first symptoms using procedure 1 or 2 twice a day for 5 days.
b. When the neurodegenerative disease has been detected, with a medical diagnostic, using procedure 1 or 2 four times a day for 10 days.
c. With an advanced neurodegenerative disease using procedure 1 or 2 eight times a day for 10 days

This therapy has been satisfactory used in PD, AD, ALS, Arthritis, Lymphedema, and Depression patients.

Pulses of chemical reagents are used widely in medicine, science and technology for characterizing, modelling and treating systems failures. Most of chemotherapy treatments use pulse methods.

The repairing of the communication failure of the immune memory is made with this invention through oxygen pulses injected in the respiratory tract. Pulses of Oxygen means that this compound is introduced in high concentration, in comparison with normal air, during a short period of time impacting the signals received and transmitted to the brain memory of the body functions. and therefore enhancing antibodies production, chemical and biochemical sensors and transmitters and practically acting as a restart button of the brain memory of the body functions. In the case of immune brain memory, these repaired signals finally produce high concentrations of lymphocytes T and B and antibodies that effectively combat the disease.

The main task of this Oxygen pulses therapy is to produce an increase of the oxygen concentration throughout the organism (alveolus, issues and brain) stimulating the brain memory of the body functions which drive to restore all the capacity of control of the body to have a healthy organism.

### DETAILED DESCRIPTION OF THE INVENTION

The pulse oxygen therapy as described therein for treating neurodegenerative diseases (PD, AD, ALS, MS, Arthritis, Lymphedema and Depression) to patients with mild, moderate, severe, or critical conditions may be applied as follow:
1. The pulse therapy treatment facilities may have sanitary oxygen supply (94-99%) with appropriate oxygen masks and water saturation flask and well isolate and well ventilate places with capacity for supporting a consumption of at least 15 l/min oxygen flow at normal conditions. Patients must have comfortable accommodation (temperature, illumination and sanitary conditions).
2. As prophylactic treatment (elder patients and/or with chronic diseases) treat patients with 6 l/min oxygen flow for 60 min once a day during 5 days.
3. As treatment for patients with mild disease (initial symptoms) treat patients with 6 l/min oxygen flow for 60 min once a day during 10 days.
4. As treatment for patients with moderate disease treat patients with 6 l/min oxygen flow for 60 min twice a day during 10 days.
5. As treatment for patients with severe disease treat patients with 6 l/min oxygen flow for 60 min four time a day during 10 days.
6. As treatment for patients with critical disease treat patients with 6 l/min oxygen flow for 60 min every 3 hours during 10 days.

Control of patients (arterial pressure, temperature) must be made by certificate medical personnel.

### Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. The following examples are illustrative only and are not a limitation on the invention described herein:

An elder patient with Parkinson Disease (72-year-old) diagnosed in 2010 (moderate conditions) was treated using method as N^{o} 4 above, and recovered after 5 days. After 12 months no AD symptoms has developed.

An elder patient with Lymphedema (73 years old) after a cancer radiotherapy treatment with severe limbs inflammation was treated using method as N^{o} 4 above, and begin recovers after 5 days, after 10 days a notable recuperation takes place. After 11 months no limbs inflammation has developed.

An elder patient with Arthritis (severe deformation in hands) (72-year-old) was treated using method as N^{o} 4 above, and begin recovers after 5 days, after 10 days a notable recuperation takes place. After 12 months no more deformation has been observed.

An elder patient with severe Depression (71 years old) was treated using method as N^{o} 4 above, and begin recovers after 5 days, after 10 days a notable recuperation takes place. After 11 months no more Depression has been detected.

### OTHERS REFERENCES

Troost et al. 1989. Clin. Neuropathol. 8:289-294.
Engelthardt et al. 1990. Arch. Neurol. 47:1210-1216.
Schiffer et al. 1996. J. Neurol. Sci. 139(suppl):27-33,
Hayashi et al. 2001 J. Neurol. Sci. 188:3-7.9-12).
Obal et al. 2001 Neuroreport. 12:2449-2452.
Sola et al. 2002 8. Eur. Neurol. 47:108-112.
Ono et al. 2001 J. Neurol. Sci. 187:27-34.
Alexianu et al. 2001 Neurology. 57:1282-1289).
Obal et al. 2001 Neuroreport. 12:2449-2452.
McGeer et al. 2002 Muscle Nerve. 26:459-47028, 29.
Moulignier et al. Reversible ALS-like disorder in HIV infection. Neurology. 57:995-1001, 2001
MacGrowen et al. An ALS-like syndrome with new HIV infection and complete response to antiretroviral therapy. Neurology. 2001, 57:1094-10.
Power et al. 2001 Adv. Virus. Res. 56:389-433.
Janssen et al. 1991, Report of a Working Group of the American Academy of Neurology AIDS Task Force. Neurology. 41:778-785.
McArthur et al. 1993 Multicenter AIDS Cohort Study. Neurology. 43:2245-2252; The Dana Consortium. 1996. Clinical confirmation of the American Academy of Neurology algorithm for HIV-1-associated cognitive/motor disorder. The Dana Consortium on Therapy for HIV Dementia and Related Cognitive Disorders. Neurology. 47:1247-1253).
Smits et al. 2000 Eur. J. Clin. Invest. 30:526-535.
Fiala et al. 2002 Eur. J. Clin. Invest. 32:360-371.
Minagar et al. 2002, The role of macrophage/microglia and astrocytes in the pathogenesis of three neurologic disorders: HIV-associated dementia, Alzheimer disease, and multiple sclerosis. J. Neurol. Sci. 202:13-23.
Pulliam et al. 1997 Lancet. 349:692-695.
Diesing et al. 2002 AIDS Reader. 12:358-368.
Glass et al. 1995 Ann. Neurol. 38:755-762.
Adamson et al. 1999 Mol. Med. 5:98-109.
Williams et al. 2002, Am. J. Pathol. 161:575-585.
Appel et al. Evidence for autoimmunity in ALS. J. Neurol. Sci. 118, (1993), pp. 169-174.
J. I. Engelhardt, &It Appel, IgG reactivity in the spinal cord and motor cortex in amyotrophic lateral sclerosis, Arch. Neurol., 47 (1990), pp. 1210-1216
Liu et al. 2000 J. Neurovirol. 6(suppl 1): S70-81
J. O. Hernández, 2021, About neurodegenerative diseases, unpublished results
Raja Bhattacharyya, Sophia E. Black, Madhura S. Lotlikar, Rebecca H. Fenn, Mehdi Jorfi, Dora M. Kovacs, Rudolph E. Axonal generation of amyloid-β from palmitoylated APP in mitochondria-associated endoplasmic reticulum membranes. Cell Reports, 2021; 35
Koos B J, Sameshima H, Power G G. Fetal breathing, sleep state, and cardiovascular responses to graded hypoxia in sheep. J Appl Physiol (1985) 1987 March; 62 (3):1033-1039.
Silver, L, Erecinska, M. Oxygen and ion concentrations in normoxic and hypoxic brain cells. In Oxygen Transport to Tissue XX, 7-15, edited by Hudetz and Bruley, Plenum Press, New York (1988).
Méndez E., Zeledón F., Zamora J., Cortés A., Rev. costarric. cardiol vol. 6 n. 1 San José, January 2004.
Ganong W. F. Review of Medical Physiology. 19th ed. California, Appleton and Lange, 1999.
Guyton A C, Hall J E. Textbook of Medical Physiology. 9th ed. Philadelphia, W. B. Saunders, 1996.
Mathews, C. K., van Holde, K. E., Ahern K. G. Biochemistry. 3rd ed. Addison Wesley, California, 2002
Heppner, Nature neuro science, 2020. Olfactory transmucosal SARS-CoV-2 invasion as a port of central nervous system entry in individuals.
Chen et al. Lancet. 2020 15-21 February; 395(10223): 507-513. Published online 2020 Jan. 30. Lymphocytopenia in patients with COVID19.
Hellerstein M., Vaccine, Vol. 6 11 Dec. 2020, 100076. What are the roles of antibodies versus a durable, high quality T-cell response in protective immunity against SARS-CoV-2? Lymphocytopenia in patients with COVID19. (Chen et al. Lancet. 2020 15-21 February; 395(10223): 507-513. Published online 2020 Jan. 30.

What is claimed is:

1. A method for treating neurodegenerative disorders such as Parkinson Disease (PD), Alzheimer's Disease (AD), Amyotrophic Lateral Sclerosis (ALS) or Motor Neuron Disease (MND) and other dementias, and Lymphedema, arthritis and depression, consisting in administering a pulse of oxygen (99-94%) through the respiratory tract using a normal Venturi oxygen mask wherein if the disease is mild would require oxygen pulses of 1 hour, 6 l/min once a day during 10 days, if it is moderate would require oxygen pulses of 1 hour, 6 l/min twice a day during 10 days, if it is severe would require oxygen pulses of 1 hour, 6 l/min, four times a day for 10 days, if it is critical would require oxygen pulses of 1 hour, 6 l/min every 3 hours during 10 days.

2. A method according to claim 1, wherein the oxygen mask is an oxygen mask of high flow high concentration with reservoir nonrebreather where if the disease is mild would require oxygen pulses of 15 minute, 6 l/min once a day during 10 days, if it is moderate would require oxygen pulses of 15 minutes, 6 l/min twice a day during 10 days, if it is severe would require oxygen pulses of 15 minutes, 6 l/min, four times a day for 10 days, if it is critical would require oxygen pulses of 15 minutes, 6 l/min every 3 hours during 10 days.
